(19) United States
(12) Patent Application Publication
(10) Pub. No.: US 2007/0134304 A1
(43) Pub. Date: Jun. 14, 2007

(54) SOLUBLE ARTICLE FOR EXFOLIATING THE SKIN

(75) Inventors: Odile Aubrun-Sonneville, Antony (FR); Dominique Bordeaux, Thiais (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/567,784

(22) Filed: Dec. 7, 2006

Related U.S. Application Data
(60) Provisional application No. 60/752,005, filed on Dec. 21, 2005.

(30) Foreign Application Priority Data
Dec. 7, 2005 (FR) 05 53753

(51) Int. Cl. A61K 9/70

(52) U.S. Cl. 424/443

(57) ABSTRACT

An article, containing a support in the form of at least one sheet comprising fibres that are water-soluble at a temperature of 0-30° C., and a composition carried by the support, comprising at least one exfoliating agent in the form of particles. The article according to the invention can be used notably for cleaning the skin and for make-up removal, for exfoliation of the skin and for dermabrasion.

### REFERENCE TO PRIOR APPLICATIONS

This application claims priority to U.S. provisional application 60/752,005 filed Dec. 21, 2005, and to French patent application 0553753 filed Dec. 7, 2005, both incorporated herein by reference.

### FIELD OF THE INVENTION

The present invention relates to articles for exfoliating the skin, comprising a water-soluble support and exfoliating particles carried by the support, and uses thereof notably in the cosmetic field for gentle exfoliation of the skin.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The use of exfoliating products for deep cleansing of the skin by a more or less abrasive effect provided by particles of micron or millimetre size is known in the cosmetic field. These particles are generally hard and of irregular shape (pumice stone, polyethylene, ground fruit stones, sand, etc.). Because of this, on the one hand, the exfoliating products containing them cannot always be used on delicate, sensitive or fine skin, as they cause redness, discomfort, burning sensations, and tightness; on the other hand, it is difficult to maintain the particles in suspension, as they sediment rapidly. This problem can be solved by adding polymers which will cause the composition to gel and thus prevent sedimentation of the particles, but there is a limited choice of polymers as it is necessary to have polymers or a combination of polymers which give a high rheological threshold to ensure good suspension of the particles; possible examples are xanthan gum combined with carboxyvinyl polymers such as the carbomers. However, the compositions gelled with such polymers are not always effective at acid pH or in the presence of electrolytes, because acids or electrolytes can destabilize in particular the ionic polymers such as the carbomers which are sensitive to such compounds. Furthermore, there is little variety in the possible textures owing to the limited choice of polymers.

### SUMMARY OF THE INVENTION

Therefore there is still a need for exfoliating products without the drawbacks of those of the prior art, and notably for exfoliating products that provide gentle exfoliation of the skin.

The present application meets this need. In fact, the inventors have found, surprisingly, that it is possible to prepare gentle exfoliating products by combining at least one support containing water-soluble fibres and an exfoliating agent in the form of particles, and thus obtain, by dissolving these articles in water or in an aqueous medium, a composition that has good exfoliating properties. These articles are moreover particularly gentle when the particles used are soft particles such as those of superabsorbent polymer.

The use of articles of this type makes it possible to remove the constraints on formulation associated with the suspension of particles. The articles obtained may also be suitable for users with delicate skin owing to the softness of the particles used, when soft particles are used. Finally, these articles make it possible to broaden the range of textures available for exfoliation, as there is no longer any incompatibility of the compounds.

### DETAILED DESCRIPTION OF THE PREFERRED EMBODIMENTS

Thus, in one of its aspects, the invention relates to an article for exfoliating the skin, comprising:
a support in the form of at least one sheet comprising fibres that are soluble in water at a temperature less than or equal to 30° C., and
a composition carried by the support, containing at least one exfoliating agent in the form of particles.

The exfoliating agent or agents can be incorporated on the support as they are, but they can also be incorporated in a mixture with other compounds. The composition carried by the support may thus only comprise the exfoliating agent or agents which in themselves constitute the composition, or it may contain the exfoliating agent or agents mixed with other compounds. This composition is preferably generally anhydrous, with “anhydrous” meaning here a composition that contains an amount of water less than or equal to 5%, and thus in the range from 0 to 5% of the weight of the composition. This composition and the exfoliating agent or agents can advantageously be in the form of powder or of granules or possibly in the form of a paste. This composition notably constitutes a cosmetic or dermatological composition.

In the present application, the expression “carried by the support” means that the composition may be either placed on the support or introduced into the cavity formed by the support when the latter comprises at least two e.g., sheets or layers forming a cavity. Of course, both situations may occur in the same article depending on the extent of overlap of the at least two supports. “On the support” includes composition located in interstices of the support.

The expression “temperature less than or equal to 30° C.” means a temperature that does not exceed 30° C. but is not less than 0° C., e.g., 0-30° C., for example ranging from more than 0° C. to 30° C., better still from 5° C. to 30° C., and even better still from 10° C. to 30° C., including 15, 20 and 25° C., and all ranges and subranges therebetween.

This article preferably constitutes a soluble or partially soluble exfoliating article. Furthermore, it presents the advantage in that it can constitute a preservative-free exfoliating article.

As pointed out below, according to a preferred embodiment of the invention, the article preferably comprises as exfoliating agent at least one superabsorbent polymer. “Superabsorbent polymer” means a crosslinked polymer which, once hydrated, forms soft beads having a number average diameter from 1 μm to 5 mm.

The terms “sheet” and “layer” are to be regarded as synonyms in the present application. The support of the present invention is preferably in the form of one or more sheets of fibres, which is different from the water-soluble thin films, which are not in the form of sheets of fibres. Compared with these water-soluble thin films, the supports based on water-soluble sheets of fibres according to the invention offer the advantage of permitting incompatible constituents to be incorporated, of being easier to use as they do not require premixing or dissolution of the components, nor heating to evaporate the solvent, the procedure also being quicker and less expensive. In addition, the supports according to the invention have the advantage of offering greater variety in the choice of shape and appearance of the article as the sheet of fibres can have variable thickness and density, giving access to a wide variety of shapes and sizes, whereas the thin film is difficult to dry if the thickness is too great, and it is fragile and difficult to manipulate if its size is too great.

According to a preferred embodiment of the invention, the article is in the form of at least two sheets defining a cavity between them, with at least one of the sheets comprising fibres that are soluble in water at a temperature less than or equal to 30° C., the cavity containing a composition containing at least one exfoliating agent.

The sheets are preferably joined together at their periphery and thus form a cavity for introduction of the composition containing the exfoliating agent.

The sheets can be formed entirely of water-soluble fibres or else one of the sheets can consist entirely of water-soluble fibres and the other sheet can consist of insoluble fibres or of both water-soluble fibres and water-insoluble fibres, or else the two sheets can consist of both soluble fibres and insoluble fibres.

According to a preferred embodiment, at least one of the sheets is constituted exclusively of water-soluble fibres.

By moistening or dissolving the article according to the invention in water or in an aqueous composition, we obtain a composition for topical, notably cosmetic or dermatological application. This composition is useful notably for application on the skin.

Thus, the invention further relates, according to another of its aspects, to an exfoliating composition obtained by dissolving, in water, an article as defined above, i.e. a composition obtained by dissolving of a support in the form of at least one sheet comprising fibres that are soluble in water at a temperature less than or equal to 30° C., said support bearing at least one exfoliating agent in the form of particles. The cosmetic or dermatological composition obtained by dissolution of the article can be obtained from a support comprising one or more layers of fibres. The temperature of dissolution of the article in water is generally room temperature (20 to 30° C.) but can be higher than room temperature if required, depending on the use envisaged.

In an example of application of the invention, the article according to the invention is brought into contact with water before it is used. Thus, it is first dissolved, before being applied to the skin of the face or of the body. It is generally dissolved completely, but it may be dissolved partially if the support includes insoluble fibres.

In another variant of application of the invention, the article according to the invention is brought into contact with the skin of the face or of the body, which has been moistened beforehand.

Thus, the invention further relates, according to another of its aspects, to a cosmetic method of skin exfoliation, comprising:
the formation of a cosmetic composition by dissolving, in water, an article comprising a support having at least one sheet comprising fibres that are soluble in water at a temperature less than or equal to 30° C. and at least one exfoliating agent in the form of particles,
application of the composition thus formed on the skin.

The invention also relates to a cosmetic method of skin exfoliation, comprising:
moistening of the skin,
applying, on the moistened skin, an article containing a support comprising at least one sheet containing fibres that are soluble in water at a temperature less than or equal to 30° C., and at least one exfoliating agent in the form of particles.

“Dissolution in water at a temperature less than or equal to 30° C.” is to be understood as meaning dissolution in water at a temperature of up to 30° C. by means of manual agitation and/or friction of the support depending on circumstances, in a space of time typically less than 5 min, preferably less than 1 min, and preferably less than 30 seconds. The invention does not exclude using water at a temperature above 30° C. for dissolving the support.

As the article according to the invention is intended for topical application, it is physiologically acceptable. “Physiologically acceptable” means an article that is compatible with keratin materials such as the skin, lips, nails, scalp and/or hair. This applies to the support, as well as to the composition and the exfoliating agents.

The article according to the invention does not contain adhesive, but it may adhere to the skin when it is moistened.

The article is flexible. “Flexible” is to be understood as meaning an article that can be compressed or can be bent without breaking, and is capable of adapting to the reliefs of the human body. A flexible article made in the form of a fibrous sheet can in certain examples of application be folded over on itself at least once without breaking in two.

This article is generally intended to be used once.

After manufacture, the article can for example be packed loose in a box or in an individual packet. If applicable, the articles are packed in a string. The articles can also be folded on themselves and interposed, so that withdrawal of one article brings the next one into a configuration so that it can be grasped easily.

Thus, the invention further relates, according to another of its aspects, to a kit comprising:
a packet,
at least one article as defined above.

In the case of a coloured composition, the article can be packed in a packet containing, if applicable, a coloured sample representative of the colour of the composition obtained after dissolution of the article, for the purpose of informing the consumer prior to purchase.

Support

The support is preferably in the form of a sheet comprising water-soluble fibres, i.e. fibres that are soluble in water at a temperature less than or equal to 30° C., preferably soluble in water at a temperature less than or equal to 20° C., i.e. having a temperature of dissolution in water ranging from more than 0° C. to 30° C., preferably from more than 0° C. to 20° C., and for example from 5° C. to 30° C., and even better from 5 to 20° C.

The support preferably is substantially non-retractable once wetted.

The support can have any shape, including any shape appropriate to the intended use, for example a rectangular, round or oval shape, and it preferably has dimensions permitting it to be grasped between at least two fingers. Thus, the support can for example be of ovoid shape about 2 to 10 cm long and about 0.5 to 4 cm wide, or of disc shape with a diameter of about 2 to 10 cm, or in the form of a square with sides of about 5 to 15 cm, or in the form of a rectangle about 5 to 15 cm long, it being understood that it can have any other shape and size suitable for the required use.

The support can form, for example, a pad, a mask, a patch, a mobcap, a glove or a finger of a glove, a sheet for cutting out, a wipe, a disc, an oval or a rectangle. Moreover, the support can have a shape that depends on the region of the body to be treated.

The support can have a flattened form or a non-flattened form, for example having the appearance of a block formed from a globular mass of compacted water-soluble fibres, incorporating a composition containing exfoliating particles.

The fibres of the support are generally interlaced to form the sheet of fibres. As stated above, “sheet comprising water-soluble fibres” means a sheet that can be constituted entirely of water-soluble fibres or a sheet that can comprise both water-soluble fibres and water-insoluble fibres, with a larger amount of soluble fibres than insoluble fibres. The sheet of fibres must comprise at least 60 wt. % of soluble fibres, preferably at least 70 wt. % and better still at least 80 wt. % relative to the total weight of the fibres. It can thus comprise, for example, more than 95 wt. %, or even more than 99 wt. % and even 100 wt. % of water-soluble fibres relative to the total weight of the fibres of the support. Thus, the support can be constituted entirely of sheets of soluble fibres or it can be constituted of sheets comprising a mixture of soluble fibres and of insoluble fibres, the insoluble fibres being, as defined in the present invention, fibres that are not soluble in water at a temperature less than or equal to 30° C. The presence of insoluble fibres may enhance the exfoliating power of the product according to the invention, because the insoluble fibres will make a contribution to exfoliation of the skin together with the particles present in the product. Furthermore, it may be beneficial to have insoluble fibres, to provide greater stability of the supports in conditions of high relative humidity.

Thus, the support can be formed from two sheets constituted of water-soluble fibres, or of one sheet constituted of water-soluble fibres and one sheet comprising both soluble fibres and insoluble fibres, or alternatively a sheet constituted of water-soluble fibres and a sheet constituted of water-insoluble fibres, or even two sheets comprising both soluble fibres and insoluble fibres. There can also be more than two sheets.

According to a preferred embodiment of the invention, the support does not have any water-insoluble fibres, but is composed solely of water-soluble fibres, so that it is completely soluble in water.

The soluble fibres can be of any soluble material that can be spun into fibres. Preferably, the water-soluble fibres are made with polyvinyl alcohol (PVA) by a method that endows them with the required solubility, and the PVA can have various degrees of polymerization.

PVA fibres that are soluble in water at a temperature less than or equal to 30° C. are marketed by the Japanese company KURARAY under the trade name KURALON K-II WN2. The method of manufacture of these fibres comprises preparation of a spinning solution by dissolving a water-soluble PVA-based polymer in a first organic solvent, spinning of the solution in a second organic solvent to obtain solidified threads and wet drawing of the threads from which the first solvent is removed and which are then dried and undergo a heat treatment. These fibres can be of roughly circular cross-section. These fibres have a tensile strength of at least 2.7 g/dtex (3 g/d). Application EP-A-0 636 716 describes such PVA-based water-soluble fibres and their method of manufacture.

The invention is not limited to the use of PVA, and it is also possible to use fibres made from other water-soluble materials provided that these materials dissolve in water of the required temperature, for example polysaccharide fibres marketed under the designation LYSORB by the company LYSAC TECHNOLOGIES, INC. or fibres based on polyholoside polymers such as glucomannan or starch.

The sheet of fibres can comprise, according to circumstances, a mixture of various fibres that are soluble in water at different temperatures (up to 30° C.).

The fibres can be composites, and they can comprise for example a core and a sheath that are dissimilar, for example formed from different grades of PVA.

When the sheet of fibres contains insoluble fibres, the latter can be of any material employed as insoluble fibres; they may for example be fibres of silk, cotton, wool, flax, cellulose extracted notably from wood, from vegetables or from algae, polyamide (Nylon®), polylactic acid, modified cellulose (rayon, viscose, acetate notably rayon acetate), poly-p-phenylene terephthalamide notably Kevlar®, of acrylic notably of methyl polymethacrylate, or of poly-2-hydroxyethyl methacrylate, of polyolefin and notably of polyethylene or of polypropylene, of glass, silica, aramid, of carbon notably in the form of graphite, of Teflon®, of insoluble collagen, of polyesters, of polyvinyl chloride or polyvinylidene chloride, polyvinyl alcohol, polyacrylonitrile, chitosan, polyurethane, polyethylene terephthalate, and fibres formed from a mixture of the compounds preferably mentioned above, such as polyamide/polyester or viscose/polyester fibres. A general description of nonwovens is given in Riedel, “Nonwoven Bonding Methods & Materials”, Nonwoven World (1987), incorporated here by reference.

In a particular example of application of the invention, the sheet of the support is a nonwoven, comprising water-soluble fibres, alone or mixed with insoluble fibres as stated above, with at most 40 wt. % of insoluble fibres relative to the total weight of the fibres constituting the sheet. Preferably, the nonwoven is constituted of water-soluble fibres, i.e. it does not contain any insoluble fibres.

When the support only has one sheet of fibres, the composition containing the exfoliating agent can be deposited on both faces of the support or on just one face, and the other face of the support can then be used for example for taking hold of the article.

When the support according to the present invention comprises two sheets, they can notably be two sheets of nonwoven, it being possible to use all the embodiments described hereunder, and the sheets may or may not contain insoluble fibres, and even one of the sheets can be constituted solely of insoluble fibres, as long as the other sheet contains soluble fibres.

According to a particular embodiment of the invention, each of the sheets is a nonwoven constituted of fibres that are soluble at a temperature less than or equal to 30° C., i.e. the sheets only comprise water-soluble fibres.

According to another embodiment, one of the sheets is completely soluble in water and is a nonwoven constituted of fibres that are soluble at a temperature less than or equal to 30° C., and the other sheet is insoluble and is a nonwoven constituted of insoluble fibres.

According to yet another embodiment, the support comprises two sheets containing fibres that are soluble or partially soluble with at most 40% of insoluble fibres relative to the total weight of the fibres, and in addition a sheet constituted of insoluble fibres, constituting an insoluble substrate. Thus, the support can comprise at least one layer of a water-insoluble substrate, i.e. only comprising insoluble fibres. In a particular example of this embodiment, the support comprises a soluble sheet of a nonwoven constituted of fibres that are soluble in water at a temperature less than or equal to 30° C., and an insoluble sheet of a nonwoven constituted of fibres that are insoluble in water.

A multilayer structure with at least one layer formed from a water-insoluble substrate can be useful for example for making an article comprising a support in the shape of a finger of a glove. The layer formed from water-soluble fibres is located on the outside of the article, and is intended to dissolve during use, after being wetted or on coming into contact with a wetted region of the body.

For the manufacture of the sheets as nonwovens, whether soluble or insoluble, all the suitable techniques for making a nonwoven material from fibres can be used. For example, the fibres can be formed by extrusion and deposited on a conveyor to form a sheet of fibres which is then consolidated by a conventional fibre bonding technique, for example needling, hot bonding, calendering or bonding by jets of hot air (called air through bonding), a technique in which the sheet passes through a tunnel into which hot air is blown. This last-preferably mentioned technique is used advantageously when the sheet is constituted of two-component fibres, for example fibres comprising at least two grades of polyvinyl alcohol (PVA), having different melting points or softening points, these fibres being for example co-extruded in such a way that the fibre is constituted of at least one first grade located in the core of the fibre and at least one second grade located at the periphery of the fibre, in the form of a sheath. Bonding of the fibres may be easier when the sheath has a lower melting point than the core.

The sheet of fibres can also be formed by carding of fibres cut to a length of 10-50 mm, then deposition of the fibres on a conveyor where the sheet can then be consolidated by a bonding technique as described above.

When the support comprises several layers, whether or not the latter are all made with water-soluble fibres, the various layers can be joined together in a variety of ways, for example by welding, gluing or stitching, and these layers can constitute, according to circumstances, one or more cavities containing one or more cosmetic or dermatological compositions or several components of one and the same cosmetic composition for mixing at the time of use. In the case of assembly by stitching, a thread that is itself water-soluble can be used, if required.

When the support comprises several nonwoven sheets, these can be joined together notably by heat sealing on their periphery so as to constitute a pad that can hold a composition containing the exfoliating agent or agents in an internal cavity.

Moreover, the support can additionally comprise at least one layer of a water-insoluble substrate, i.e. comprising only insoluble fibres, and in this embodiment, the support comprises a sheet of a nonwoven constituted of fibres that are soluble in water at a temperature less than or equal to 30° C., and a sheet of a nonwoven constituted of water-insoluble fibres.

According to another aspect of the invention, the support is not provided with adhesive, notably pressure-sensitive adhesive.

The density of the support may depend on the applications. The support can have, for example, a density less than or equal to 0.1 g/cm³ or alternatively greater than 0.1 g/cm³. According to a preferred embodiment of the invention, the support has a density less than or equal to 0.1 g/cm³, and better still in the range from 0.01 g/cm³ to 0.1 g/cm³, including 0.02, 0.04, 0.06, and 0.08 g/cm³ and all subranges and values therebetween, which can provide a very aerated support, which accordingly dissolves more readily in water.

The composition containing at least one exfoliating agent preferably represents between 10 and 1000 wt. % relative to the weight of the support, and more preferably between 10 and 500 wt. % relative to the weight of the support, where “weight of the support” means in this case the weight of the support alone, without the weight of the composition containing the exfoliating agent. If the composition only contains exfoliating agents, it is the latter that can represent between 10 and 1000 wt. % relative to the weight of the support, and preferably between 10 and 500 wt. % relative to the weight of the support.

Exfoliating Agents

The article according to the invention contains at least one exfoliating agent in the form of particles. These particles can be hard or soft.

As stated above, the composition carried by the support can be constituted of one or more exfoliating agents, the term “constituted” meaning that there is no other compound, and the exfoliating agent or agents can be incorporated directly on the support. According to another embodiment, the composition comprises one or more exfoliating agents mixed with other compounds, and the composition is prepared by mixing the various compounds before they are incorporated on the support.

The exfoliating agents used in the product according to the invention can be selected from all of the exfoliating particles of mineral, vegetable or organic origin, usually employed in the cosmetic field, and for example from water-swellable pulverulent polymers (powder or beads), polyethylene particles (beads or powders), jojoba spheres, ground shells of fruit stones, pumice stone, glass beads, aluminium oxide, and mixtures thereof.

Pulverulent polymers that swell in water after hydration can notably be in the form of beads having an average diameter in the range from about 1 μm to about 5000 μm (5 mm), and preferably in the range from 10 μm to 5 mm. As water-swellable pulverulent polymers, we may preferably mention in particular the superabsorbent polymers, such as particles of crosslinked sodium polyacrylate, for example those marketed under the designations Octacare X100, X110 and RM100 by the company Avecia, those marketed under the designations Flocare GB300 and Flosorb 500 by the company SNF, starches grafted with sodium polyacrylate (INCI name: Sodium polyacrylate starch) such as those marketed under the designations Sanfresh ST-100C., ST100MC., IM-300MC by the company Sanyo Chemical Industries, hydrolysed starches grafted with acryloacrylamide/sodium acrylate copolymer (INCI name: Starch/acrylamide/sodium acrylate copolymer), such as those marketed under the designations Water Lock A-240, A-180, B-204, D-223, A-100, C-200, D-223, G-400 by the company Grain Processing, and mixtures thereof.

The exfoliating agents can also be selected from polyethylene particles, jojoba spheres such as those marketed under the designations Florasome by the company Floratech, ground shells of fruit stones, pumice stone (INCI name: pumice) such as that marketed under the designation Ponce 3/B by the company Eyraud, glass beads, aluminium oxide such as that marketed under the designation DERMAGRAIN 900 by the company Marketech International.

According to a preferred embodiment of the invention, the article comprises at least one superabsorbent polymer alone or in mixture with at least one exfoliating agents selected from polyethylene particles, jojoba spheres, ground shells of fruit stones, pumice stone, glass beads, aluminium oxide.

The exfoliating agents are present in variable amounts depending on the result required. Their amount depends also from the used exfoliating agent, and it preferably represents from 2 to 100 wt. % relative to the weight of the composition, and better still from 10 to 100 wt. % relative to the weight of the composition. When a superabsorbent polymer is present as exfoliating agent, its amount preferably represents from 2 to 30 wt. % relative to the weight of the composition, and better from 2 to 25 wt. % relative to the weight of the composition carried by the support.

The composition containing the exfoliating agent is preferably an anhydrous pulverulent or pasty composition. Thus, preferred compositions that can be used in the invention can be for example:
lyophilized or atomized emulsions, such as those described in document FR-A-2,727,312 or those based on modified starch described in document EP-A-0 938 892. These emulsions are obtained by lyophilization or atomization of an O/W emulsion containing a pulverulent phase, giving milks or creams on mixing with water during use,
foaming compositions in the form of powders, containing pulverulent surfactants, such as those based on starch, described in document EP-A-0 925 777, giving mousse on mixing with water during use,
oil-free pulverulent compositions, containing for example hydrophilic gelling agents and/or surfactants, and giving gels or lotions on mixing with water during use,
compositions obtained by simple mixing of the constituents, the latter preferably being in the form of powder.

The composition can if necessary from an end use point of view contain a certain amount of water at the moment of its impregnation on the support. However, to prevent its premature dissolution, the water introduced on the support during its impregnation must be removed by the means conventionally employed for the dehydration of compositions containing water, for example heating. However, the composition can contain a certain amount of water which is generally bound water and which may originate notably from hygroscopic raw materials that contain water, such as starches. The final amount of water in the composition present on the article is at most 20 wt. % and preferably at most 10 wt. % relative to the total weight of the composition.

When the composition must be deposited on the support by the user, the composition and the support can be supplied in the form of a kit. The composition is for example supplied in a sufficient amount for distributing a plurality of doses thereof on a set of supports that are to be used successively.

Additives

The composition containing one or more exfoliating agents can contain, depending on the final use of the product, compounds other than the exfoliating agents. These additives can notably be anhydrous or in solid form (powder). They can be selected notably from those generally employed in the cosmetic and dermatological fields, for example sequestering agents, perfumes, antioxidants, active agents, preservatives, colouring matter (such as pigments and hydrophilic dyes), mineral fillers and/or organic fillers, lipophilic compounds, and water-soluble gelling agents. Of course, a person skilled in the art will make sure that any of these additives and/or their amounts are selected in such a way that the advantageous properties that are intrinsic to the composition according to the invention will not, or substantially not, be adversely affected by the addition or additions envisaged.

We may preferably mention, as examples of water-soluble gelling agents, cellulose derivatives such as carboxymethylcellulose, xanthan gums, scleroglucan, gellan, rhamsan, alginates, gum karaya, modified or unmodified guar gums, and their derivatives such as hydroxypropylguar, modified starches and mixtures thereof.

The following may be preferably mentioned as examples of modified starches:
crosslinked and acetylated maize starch sold by the company Cerestar under the name C* Flo 06205,
amylopectin/amylose modified starch, sold under the trade name Remyline AP by the company Remy,
modified, precooked wheat starch, sold under the trade name Midsol Krisp by the company Midwest Grain Products,
modified, refined wheat starch, sold under the trade name Midsol Adhere by the company Midwest Grain Products,
modified wheat starch powder, sold under the trade name Midsol 35 by the company Midwest Grain Products,
modified potato starch, sold under the trade name Perfectagel MPT by the company Avebe,
and mixtures thereof.

The amount of water-soluble gelling agent in the composition can range for example from 0.1 to 10 wt. %, better still from 0.1 to 5 wt. % and even better still from 0.1 to 3 wt. % relative to the total weight of the composition.

Furthermore, the composition can contain one or more active agents. The active agents can be selected notably from the keratolytic agents, moisturizers, soothing agents and antimicrobials.

We may preferably mention, as moisturizers, the polyols such as glycerol; compounds acting on the barrier function, in order to maintain the hydration of the stratum corneum, or occlusive compounds, in particular the ceramides, sphingoid-based compounds, lecithins, glycosphingolipids, phospholipids, cholesterol and its derivatives, phytosterols (stigmasterol, β-sitosterol, campesterol), essential fatty acids, 1,2-diacylglycerol, 4-chromanone, the pentacyclic triterpenes such as ursolic acid, vaseline and lanolin; compounds that directly increase the water content of the stratum corneum, such as threalose and its derivatives, hyaluronic acid and its derivatives, glycerol, pentanediol, sodium pidolate, serine, xylitol, sodium lactate, glycerol polyacrylate, ectoin and its derivatives, chitosan, oligosaccharides and polysaccharides, cyclic carbonates, N-lauroyl pyrrolidone carboxylic acid, and N-α-benzoyl-L-arginine; and mixtures thereof.

The following may be preferably mentioned as keratolytic agents: the β-hydroxy acids, in particular salicylic acid and its derivatives (including n-octanoyl-5-salicylic acid); the α-hydroxy acids, such as glycolic, citric, lactic, tartaric, malic or mandelic acids, and mixtures thereof.

The following may be preferably mentioned as examples of soothing agents that can be used in the composition according to the invention: pentacyclic triterpenes and plant extracts (e.g. Glycyrrhiza glabra) containing them, such as β-glycyrrhetinic acid and its salts and/or its derivatives (glycyrrhetinic acid monoglucuronide, stearyl glycyrrhetinate, 3-stearoyloxyglycyrrhetic acid), ursolic acid and its salts, oleanolic acid and its salts, betulinic acid and its salts, extracts from plants such as Paeonia suffruticosa and/or lactiflora, Laminaria saccharina, Boswellia serrata, Centipeda cunninghamii, Helianthus annuus, Linum usitatissimum, Cola nitida, Epilobium angustifolium, Aloe vera, Bacopa monieri, the salts of salicylic acid and in particular zinc salicylate, Canola oil, bisabolol and chamomile extracts, allantoin, Sepivital EPC (phosphoric diester of vitamin E and C) from Seppic, unsaturated omega-3 oils such as musk rose oil, blackcurrant oil, ecchium oil or fish oil, plankton extracts, capryloyl glycine, Seppicalm VG (sodium palmitoylproline and nymphea alba) from Seppic, tocotrienols, piperonal, an extract of clove, phytosterols, cortisone, hydrocortisone, indomethacin and betamethasone.

The following may be preferably mentioned as examples of antimicrobials: 2,4,4′-trichloro-2′-hydroxydiphenyl ether (or triclosan), 3,4,4′-trichlorocarbanilide (or triclocarban), phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulphaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, octopirox, octoxyglycerol, octanoylglycine, caprylyl glycol, 10-hydroxy-2-decanoic acid, dichlorophenyl imidazole dioxolan and its derivatives described in patent WO-A-93/18743, farnesol, phytosphingosines and their mixtures.

As vitamins, water-soluble or fat-soluble vitamins or provitamins can be used, such as, for example, vitamins A (retinol), C (ascorbic acid), B3 or PP (niacinamide), B5 (panthenol), B6 or pyridoxine, E (tocopherol), K1, beta-carotene, and the derivatives of these vitamins and notably their esters, and mixtures thereof.

The composition containing the exfoliating agent can also contain one or more lipophilic compounds, fats and notably oils, or oily active agents. The amount of lipophilic compound can range for example from 0.1 to 50 wt. % relative to the total weight of the composition carried by the support.

It is possible to use any kind of oil and fat familiar to a person skilled in the art, such as oils of vegetable origin (for example jojoba, avocado, sesame, sunflower, maize, soya, safflower, grapeseed oils), mineral oils (for example vaseline, isoparaffins, optionally hydrogenated), synthetic oils (for example isopropyl myristate, cetearyl octanoate, polyisobutylene, ethyl-hexyl palmitate or myristate, alkyl benzoates), volatile or non-volatile silicone oils, and fluorinated or fluorosiliconized oils, as well as mixtures of these oils.

The following may be preferably mentioned as other fats: fatty alcohols such as stearyl alcohol, cetyl alcohol and their mixture (cetearyl alcohol), fatty acids, gums, for example silicone gums such as the PDMS mixture with hydroxylated alpha-omega groups/PDMS 5 cSt (12/88) sold under the designation DC 1503 by the company Dow Corning, and the lipophilic gelling agents such as bentone.

The composition can also contain one or more foaming surfactants, preferably of pulverulent form, i.e. in the form of powder. The foaming surfactants that can be used are all those usually employed in the cosmetic field, and these surfactants can be anionic, non-ionic, cationic, amphoteric or zwitterionic.

The preferred surfactants are those in powder form, for example sodium lauryl sulphate such as the product marketed under the designation Empicol LZ D by the company Allbright & Wilson or under the designation Tensopol USP97 by the company Tensachem; cocamidopropylbetaine such as the product marketed under the designation Tegobetain CK D by the company Degussa; sodium lauroyl glutamate such as the product marketed under the designation Amisoft LS 11 by the company Ajinomoto; monosodium myristoyl glutamate such as the product marketed under the designation Acylglutamate MS 11 by the company Ajinomoto; the mixture of sodium laureth sulphate and silica, marketed under the designation Texapon KE 2713 by the company Cognis; disodium cocamido MEA-sulphosuccinate such as the product marketed under the designation Mackanate CM 100 by the company MacIntyre; sodium methyl cocoyl taurate, such as the product marketed under the designation Tauranol WSP by the company Finetex; sodium decyl d-galactoside uronate such as the product marketed under the designation Sodium decyl d-galactoside uronate by the company Ard-Soliance; lauroyl methyl beta-alanine (acid form) marketed under the designation LMA-H by the company Mitsui Toatsu; n-lauroyl-n-hydroxyethyl-beta-alanine marketed under the designation LHEA by the company Mitsui Toatsu; sodium cocoyl glycinate marketed under the designation Amilite GCS-11(F) by the company Ajinomoto; sodium cocoyl isethionate such as the product marketed under the designation Jordapon CI P by the company BASF; sodium lauryl sulphoacetate, such as the product marketed under the designation Lathanol LAL powder by the company Stepan; potassium myristate such as the product marketed under the designation potassium myristate (DUB MK) by the company Stearineries Dubois; potassium laurate such as the product marketed under the designation potassium laurate (DUB LK) by the company Stearineries Dubois, and sucrose laurate such as the product marketed under the designation Grilloten LSE 87 by the company Degussa.

The article according to the invention finds application notably for cleaning and exfoliation of the skin (scrub) including dermabrasion, and it can constitute notably a skin cleanser, an exfoliating article, and a make-up remover.

The examples below serve to illustrate the invention, but without limiting its scope. The amounts stated are percentages by weight unless stated otherwise, and they correspond, unless stated otherwise, to the amount of raw material and not to the amount of active substance.

The names of the compounds used are given as INCI name, as chemical name or as trade name.

### EXAMPLES

The product used in the examples was made with a support of PVA-based Kuralon K-II WN2 fibres. It was obtained by heat-sealing two layers, with a weight of 80 g/m², at their periphery.

The product was in the form of a disc with a diameter of 3 cm, having a cavity in which the composition was placed. In the case of Example 1, the amount of composition placed in the cavity was 0.1 g; in Examples 2 and 4, the amount was 0.3 g, and in Example 3 it was 0.5 g.

| | Example 1 | Example 2 | Example 3 | Example 4 |
|---|---|---|---|---|
| | according | according | according | according |
| | to the | to the | to the | to the |
| | invention | invention | invention | invention |
| Sodium polyacrylate | 5 | 10 | — | — |
| (1) | | | | |
| Sorbitol | — | 50 | — | — |
| (moisturizer) | | | | |
| Carboxymethyl- | — | 40 | — | — |
| cellulose (2) | | | | |
| Potassium myristate | — | — | — | 90 |
| (3) | | | | |
| Pumice stone | — | — | — | 10 |
| Polyethylene powder | 50 | — | 10 | — |
| (4) | | | | |
| Modified starch (5) | — | — | 22.5 | — |
| Vaseline oil | — | — | 67.5 | — |
| USE | Scrub | Gentle | Make-up | Foaming |
| | | exfoliation | remover/ | scrub |
| | | for dry and | exfoliating | |
| | | sensitive | cream | |
| | | skin | | |

(1) Octacare MS100 (Avecia)
(2) Blanose 9M31F (Hercules)
(3) Potassium myristate (DUB MK) (Stéarineries Dubois)
(4) Microthene MN 727 (Equistar)
(5) C* Flo 06205 (Cerestar)

Procedures:
Examples 1, 2 and 4 were obtained by mixing the powders, and then placing the mixture in the cavity of the support, which was then closed by heat-sealing.
Example 3 was a powder obtained by atomization of an emulsion as described in EP-A-0 938 892. As in the other examples, the powder was placed in the cavity of the support, which was then closed by heat-sealing.
   Manner of Use:

In all the examples, the article is first wetted with an amount of water greater than or equal to 2 ml before being applied to the area to be treated.

In Example 3, the mousse is preferably produced in the hand by successive additions of water according to the amount of mousse required. It is then applied to the face.

In the other examples, the moistened article can be applied directly to the area to be treated, moistened or not, or alternatively cut in the hand before being applied to the area to be treated.

In all cases, the skin is then rinsed.

The above written description of the invention provides a manner and process of making and using it such that any person skilled in this art is enabled to make and use the same, this enablement being provided in particular for the subject matter of the appended claims, which make up a part of the original description and including an article, in particular a cosmetic or dermatological article, comprising a support in the form of at least one sheet comprising fibres that are soluble in water at a temperature less than or equal to 30° C., and a composition carried by the support, containing at least one exfoliating agent in the form of particles.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. An article, comprising:
a support in the form of at least one sheet comprising fibres that are water-soluble at a temperature of 0-30° C., and
a composition carried by the support, comprising at least one exfoliating agent in the form of particles.

2. The article according to claim 1, wherein the fibres that are soluble in water at a temperature of 0-30° C. are made with polyvinyl alcohol.

3. The article according to claim 1, wherein at least one sheet comprising fibres is a nonwoven.

4. The article according to claim 1, wherein at least one sheet comprising fibres further comprises water-insoluble fibres.

5. The article according to claim 4, wherein the amount of water-insoluble fibres is at most 40 wt. % relative to the total weight of the fibres of the support.

6. The article according to claim 1, wherein the support comprises at least two sheets, each containing fibres that are soluble in water at a temperature of 0-30° C.

7. The article according to claim 1, wherein the support comprises at least two sheets comprising fibres defining a cavity between them, with at least one of the sheets comprising fibres that are soluble in water at a temperature of 0-30° C., and wherein the cavity containing the composition containing at least one exfoliating agent in the form of particles.

8. The article according to claim 7, wherein at least two sheets of fibres are nonwovens.

9. The article according to claim 8, wherein one of the sheets is a nonwoven constituted of fibres that are soluble in water at a temperature less than or equal to 30° C., and another of the sheets is a nonwoven constituted of water-insoluble fibres.

10. The article according to claim 7, wherein at least two sheets are joined together at their periphery.

11. The article according to claim 10, wherein the sheets are heat-sealed.

12. The article according to claim 1, wherein the support is completely soluble in water.

13. The article according to claim 1, wherein the amount of exfoliating agent ranges from 2 to 100 wt. % relative to the total weight of the composition.

14. The article according to claim 1, wherein the exfoliating agent is at least one selected from water-swellable polymers, polyethylene particles, jojoba spheres, ground shells of fruit stones, pumice stone, glass beads, aluminium oxide, and mixtures thereof.

15. The article according to claim 1, wherein the exfoliating agent is a water-swellable polymer selected from superabsorbent polymers.

16. The article according to claim 15, wherein the superabsorbent polymer is selected from the particles of crosslinked sodium polyacrylate, starches grafted with sodium polyacrylate, hydrolysed starches grafted with acryloacrylamide/sodium acrylate copolymer, and mixtures thereof.

17. A composition obtained by dissolving the article according to claim 1 in water.

18. A method of skin exfoliation, comprising:
forming a composition by dissolving, in water, a support in the form of at least one sheet comprising fibres that are soluble in water at a temperature of 0-30° C. and at least one exfoliating agent in the form of particles, and
applying the composition on the skin.

19. A method of skin exfoliation, comprising:
moistening the skin,
applying, on the moistened skin, the article according to claim 1.

20. Kit comprising:
a packet,
at least one article according to claim 1.
